(19) United States
(12) Patent Application Publication
(10) Pub. No.: US 2024/0164673 A1
(43) Pub. Date: May 23, 2024

(54) HEALTH STATUS MANAGEMENT SYSTEM, INFORMATION PROCESSING APPARATUS, AND INFORMATION PROCESSING METHOD

(71) Applicants: Kazuki Funahashi, KANAGAWA (JP); Toshiaki Tokita, KANAGAWA (JP); Masako Yoshii, KANAGAWA (JP); Naoki Sakai, KANAGAWA (JP)

(72) Inventors: Kazuki Funahashi, KANAGAWA (JP); Toshiaki Tokita, KANAGAWA (JP); Masako Yoshii, KANAGAWA (JP); Naoki Sakai, KANAGAWA (JP)

(73) Assignee: Ricoh Company, Ltd., Tokyo (JP)

(21) Appl. No.: 18/502,374

(22) Filed: Nov. 6, 2023

(30) Foreign Application Priority Data
Nov. 22, 2022 (JP) 2022-186575
Mar. 3, 2023 (JP) 2023-032781

(51) Int. Cl. A61B 5/16; A61B 5/00; A61B 5/024; G16H 10/20

(52) U.S. Cl. CPC A61B 5/165; A61B 5/02416; A61B 5/742; A61B 5/746; G16H 10/20

(57) ABSTRACT

A health status management system includes circuitry to measure a pulse wave of a user to obtain objective evaluation data for objective evaluation in relation to a mental health status, obtain one or more answers of the user to one or more questions related to the mental health status as subjective evaluation data for subjective evaluation in relation to the mental health status, control a period of measuring the pulse wave and a period of performing the subjective evaluation to overlap with each other, control a start time of the period of measuring the pulse wave to be earlier than a start time of the period of performing the subjective evaluation, transmit, to additional circuitry, the objective evaluation data and the subjective evaluation data, and display, on a screen, information on the objective evaluation and information on the subjective evaluation.

### CROSS-REFERENCE TO RELATED APPLICATIONS

This patent application is based on and claims priority pursuant to 35 U.S.C. § 119(a) to Japanese Patent Application Nos. 2022-186575, filed on Nov. 22, 2022, and 2023-032781, filed on Mar. 3, 2023, in the Japan Patent Office, the entire disclosure of which is hereby incorporated by reference herein.

### BACKGROUND

### Technical Field

The present disclosure relates to a health status management system, an information processing apparatus, and an information processing method.

### Related Art

A technique for objectively evaluating the mental health status (mental status) of an employee of a company by measuring biometric data related to, for example, the autonomic nervous system is known. In the related art, a result of objective evaluation related to a mental health status based on biometric information and a result of subjective evaluation related to the mental health status based on answers to questions related to the health status are presented to users.

### SUMMARY

An embodiment of the present disclosure includes a health status management system including circuitry to measure a pulse wave of a user to obtain objective evaluation data for objective evaluation in relation to a mental health status, obtain one or more answers of the user to one or more questions related to the mental health status as subjective evaluation data for subjective evaluation in relation to the mental health status, control a period of measuring the pulse wave and a period of performing the subjective evaluation to overlap with each other, control a start time of the period of measuring the pulse wave to be earlier than a start time of the period of performing the subjective evaluation, transmit, to additional circuitry, the objective evaluation data and the subjective evaluation data, and display, on a screen, information on the objective evaluation and information on the subjective evaluation.

An embodiment of the present disclosure includes an information processing apparatus including circuitry to measure a pulse wave of a user to obtain objective evaluation data for objective evaluation in relation to a mental health status of the user, obtain an answer of the user to a question related to the mental health status as subjective evaluation data for subjective evaluation in relation to the mental health status, control a period of measuring the pulse wave and a period of performing the subjective evaluation to overlap with each other, and control a start time of the period of measuring the pulse wave to be earlier than or same as a start time of the period of performing the subjective evaluation.

An embodiment of the present disclosure includes an information processing method including measuring a pulse wave of a user to obtain objective evaluation data for objective evaluation in relation to a mental health status, obtaining an answer of the user to a question related to the mental health status as subjective evaluation data for subjective evaluation in relation to the mental health status, controlling a period of measuring the pulse wave and a period of performing the subjective evaluation to overlap with each other, and controlling a start time of the period of measuring the pulse wave to be earlier than or same as a start time of the period of performing the subjective evaluation.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram illustrating a health status management system according to some embodiments of the present disclosure;

FIG. 2 is a block diagram illustrating a hardware configuration of each of an information processing apparatus, an analysis apparatus, and a terminal apparatus according to some embodiments of the present disclosure;

FIG. 3 is a block diagram illustrating a functional configuration of the health status management system according to some embodiments of the present disclosure;

FIG. 4 is a flowchart of an overall process performed by the health status management system according to some embodiments of the present disclosure;

FIG. 5 is a flowchart of a process for obtaining objective evaluation data and subjective evaluation data according to some embodiments of the present disclosure;

FIG. 6 is a diagram illustrating a screen for starting pulse wave measurement according to some embodiments of the present disclosure;

FIG. 7 is a diagram illustrating a screen for checking pulse wave measurement according to some embodiments of the present disclosure;

FIG. 8 is a diagram illustrating a screen for displaying a warning according to some embodiments of the present disclosure;

FIG. 9 is a diagram illustrating a screen for starting analyzing a pulse wave according to some embodiments of the present disclosure;

FIG. 10 is a diagram illustrating a screen for presenting a question for subjective evaluation according to some embodiments of the present disclosure;

FIG. 11 is a flowchart of a process for measuring a pulse wave according to some embodiments or the present disclosure;

FIG. 12 is a flowchart of a process for determining a mental health status according to some embodiments of the present disclosure; and

FIG. 13 is a flowchart of a process for calculating an objective indicator according to some embodiments of the present disclosure.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

A health status management system, an information processing apparatus, an information processing method, and a program according to one or more embodiments of the present disclosure are described below with reference to the attached drawings.

### First Embodiment

System Configuration

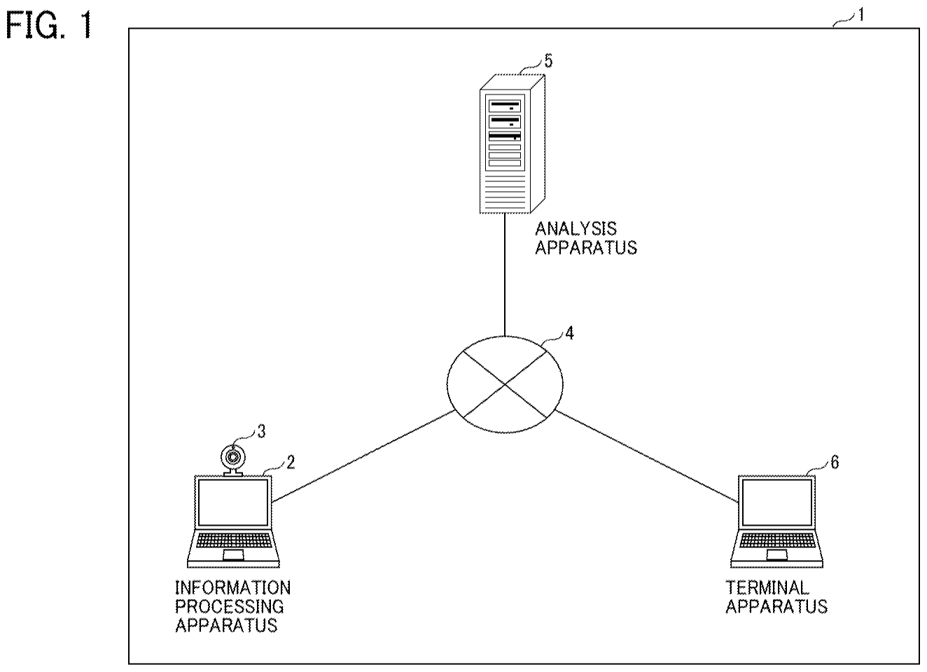

FIG. 1 is a diagram illustrating a health status management system according to an embodiment of the present disclosure. The health status management system 1 includes, for example, an information processing apparatus 2, an analysis apparatus 5, and a terminal apparatus 6 that are connected to a communication network 4 such as the Internet. In the health status management system 1, the analysis apparatus 5 determines a mental health status of a user (first user) based on objective evaluation data and subjective evaluation data obtained by the information processing apparatus 2, and reports a determination result to the user and an administrator (second user) who manages, supervises, or observes the health status of the user. By operating the terminal apparatus 6, the administrator can check the determination result that indicates the mental health status of the user and is reported from the analysis apparatus 5. The user is, for example, an employee of a company, and operates the information processing apparatus 2 in order to evaluate his or her mental health status in an office of the company or in a remote work place such as a home or a place at which the user works or visits. The administrator is, for example, a supervisor or a manager of the user, a person in charge of human resources of the company to which the user belongs, or an occupational health staff such as an occupational medicine or a public health nurse.

The information processing apparatus 2 is, for example, a laptop personal computer, a desktop personal computer, a tablet terminal, or a smartphone, and includes a measurement device 3 to measure objective evaluation data related to a mental health status of the user. The measurement device 3 is, for example, a camera, a pulse wave sensor, or an electrocardiogramsor, and measures the pulse wave of the user. The measurement device 3 may be built in the information processing apparatus 2 or may be externally connected to the information processing apparatus 2. The external connection may be wired connection using, for example, a Universal Serial Bus (USB) cable, or may be wireless connection using, for example, BLUETOOTH (registered trademark). In order to evaluate the mental health status of the user, the information processing apparatus 2 performs objective evaluation to obtain the pulse wave of the user as objective evaluation data, and performs subjective evaluation to obtain one or more answers of the user to one or more questions related to a mental health status as subjective evaluation data. Further, the information processing apparatus 2 transmits the objective evaluation data and the subjective evaluation data, which are obtained, to the analysis apparatus 5. The information processing apparatus 2 causes a period of performing the objective evaluation and a period of performing the subjective evaluation to overlap with each other, and the total evaluation period can be reduced or shortened, accordingly.

The analysis apparatus 5 determines the mental health status of the user based on the objective evaluation data and the subjective evaluation data, which are received from the information processing apparatus 2, and stores and manages the objective evaluation data, the subjective evaluation data, and the determination result in a storage device. Further, the analysis apparatus 5 notifies the user of information on the determination result and transmits the information on the determination result to the information processing apparatus 2. Further, the analysis apparatus 5 notifies the administrator of the information on the determination result, and transmits the information on the determination result to the terminal apparatus 6.

The terminal apparatus 6 is, for example, a laptop personal computer, a desktop personal computer, a tablet terminal, or a smartphone, operated by the administrator who manages, supervises, or observes the health status of the user. The terminal apparatus 6 displays the determination result on a screen based on the information received from the analysis apparatus 5.

The system configuration of the health status management system 1 illustrated in FIG. 1 is merely an example. For example, each of the number of information processing apparatuses 2 and the number of terminal apparatuses 6 may be any number. Further, the communication network 4 may include, for example, a connection section by mobile communication or wireless communication such as wireless Local Area Network (LAN).

An application to be executed to implement each function or unit in the information processing apparatus 2 and the terminal apparatus 6 may be a native application that operates on an operating system (OS) or a web application that operates on a web browser.

Hardware Configuration

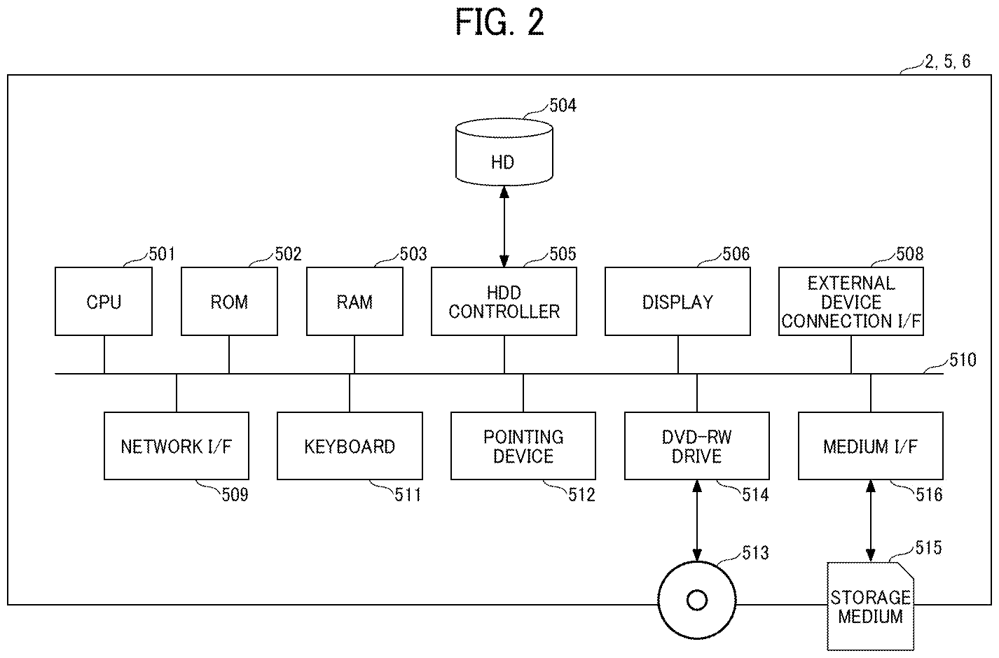

FIG. 2 is a block diagram illustrating a hardware configuration of each of the information processing apparatus 2, the analysis apparatus 5, and the terminal apparatus 6 according to the present embodiment. As illustrated in FIG. 2, each of the information processing apparatus 2, the analysis apparatus 5, and the terminal apparatus 6 is implemented by a computer, and includes a central processing unit (CPU) 501, a read only memory (ROM) 502, a random access memory (RAM) 503, a hard disk (HD) 504, a hard disk drive (HDD) controller 505, a display 506, an external device connection interface (I/F) 508, a network I/F 509, a bus line 510, a keyboard 511, a pointing device 512, a digital versatile disc rewritable (DVD-RW) drive 514, and a medium I/F 516.

The CPU 501 performs overall control of a corresponding one of the information processing apparatus 2, the analysis apparatus 5, and the terminal apparatus 6. The ROM 502 stores programs such as an initial program loader (IPL) to boot the CPU 501. The RAM 503 is used as a work area for the CPU 501. The HD 504 stores various data such as a program. The HDD controller 505 controls reading and writing of various data from and to the HD 504 under control of the CPU 501. The display 506 displays various information such as a cursor, a menu, a window, a character, or an image. The external device connection I/F 508 is an interface that connects to various external apparatuses (devices). Examples of the external devices include, but are not limited to, a USB memory and a printer. The network I/F 509 is an interface for data communication via the communication network 4. The bus line 510 is, for example, an address bus or a data bus, which electrically connects the components illustrated in FIG. 2, such as the CPU 501.

The keyboard 511 is an example of an input device provided with multiple keys used to input characters, numerals, or various instructions. The pointing device 512 is an example of an input device that allows a user to select or execute various instructions, select an item for processing, or move a cursor being displayed. The DVD-RW drive 514 reads and writes various data from and to a DVD-RW 513 that is serves as a removable storage medium (recording medium). The DVD-RW drive 514 is not limited to the DVD-RW and may be a digital versatile disc-recordable (DVD-R). The medium I/F 516 controls reading and writing (storing) of data from and to a storage medium 515, such as a flash memory.

Functions

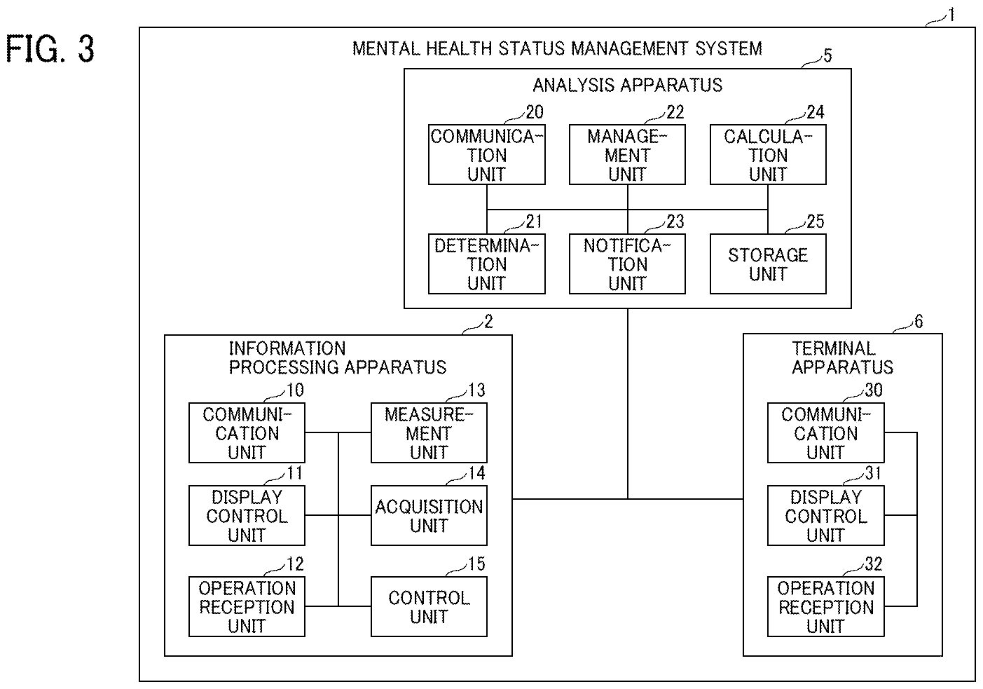

FIG. 3 is a block diagram illustrating a functional configuration of the health status management system 1 according to the present embodiment. The information processing apparatus 2 includes a communication unit 10, a display control unit 11, an operation reception unit 12, a measurement unit 13, an acquisition unit 14, and a control unit 15. Each of the functional units is implemented by the CPU 501 executing an instruction included in one or more programs installed in the information processing apparatus 2.

The communication unit 10 is a communication function of the information processing apparatus 2, and transmits and receives information to and from, for example, the analysis apparatus 5 via the communication network 4. For example, the communication unit 10 transmits the objective evaluation data and the subjective evaluation data that are related to the mental health status of the user to the communication unit 20 of the analysis apparatus 5. Further, the communication unit 10 receives the result of the mental health status from the communication unit 20 of the analysis apparatus 5.

The display control unit 11 causes a screen of the information processing apparatus 2 to display a screen of an application for evaluating a mental health status to evaluate the mental health status of the user and a message related to the evaluation. For example, the display control unit 11 causes the screen of the information processing apparatus 2 to display a warning (alert) for notifying the user of a failure to measure the pulse wave.

The operation reception unit 12 receives an operation such as inputting a character or pressing a button performed by the user via a keyboard or a pointing device of the terminal apparatus 6. For example, the operation reception unit 12 receives an operation (for example, pressing an answer button or inputting an answer) for making an answer to a question related to the mental health status by the user.

The measurement unit 13 measures and acquires the pulse wave of the user as objective evaluation data related to the mental health status of the user by using the measurement device 3 such as a camera. Further, the measurement unit 13 checks whether the pulse wave is appropriately measurable.

The acquisition unit 14 obtains an answer (subjective evaluation data) from the user to a question related to the mental health status. The question is presented to the user.

The control unit 15 controls transition of processes performed by the information processing apparatus 2. Further, the control unit 15 sets and controls the start times and the end times of the period of performing the objective evaluation (objective-evaluation period), a period of measuring the pulse wave (pulse-wave-measurement period), a period of checking whether the pulse wave is measurable (checking period), a period of analyzing the pulse wave (pulse-wave-analysis period), and the period of performing the subjective evaluation (subjective-evaluation period). Details of the control executed by the control unit 15 are described later.

The analysis apparatus 5 includes a communication unit 20, a determination unit 21, a management unit 22, a notification unit 23, and a calculation unit 24. Each of the functional units is implemented by the CPU 501 executing an instruction included in one or more programs installed in the analysis apparatus 5. A storage unit 25 can be implemented by, for example, a storage device such as the HD 504 included in the analysis apparatus 5.

The communication unit 20 is a communication function of the analysis apparatus 5, and transmits and receives information to and from, for example, the information processing apparatus 2 or the terminal apparatus 6 via the communication network 4.

The determination unit 21 determines the mental health status of the user based on the objective evaluation data and the subjective evaluation data received from the information processing apparatus 2. Details of the determination method are described later.

The management unit 22 integrates information related to mental health the status for each of all users to centrally manage the information. For example, the management unit 22 causes the storage unit 25 to store, for example, information identifying a user (for example, name and user identifier (ID)) and information on date and time (evaluation execution date and time) in association with the objective evaluation data and the subjective evaluation data received from the information processing apparatus 2 and the determination result determined by the determination unit 21.

The notification unit 23 notifies the determination result determined by the determination unit 21 to the user and the administrator who manages, supervises, or observes the health status of the user. Further, the notification unit 23 notifies information to be notified to the administrator who manages, supervises, or observes the health status of the user based on the determination result determined by the determination unit 21. Details of the method for notifying information and the information to be notified are described later.

The calculation unit 24 calculates an objective indicator that is an indicator indicating the mental health status, based on the objective evaluation data obtained by measuring the pulse wave. Further, the calculation unit 24 calculates a subjective indicator that is an indicator indicating the mental health status, based on the subjective evaluation data. Details of the calculation method for the objective indicator and the subjective indicator are described later.

The storage unit 25 stores the objective evaluation data, the subjective evaluation data, and the determination result of the mental health status of the user determined by the determination unit 21, which are related to the mental health status of the user, in association with, for example, the information identifying the user (for example, name and user ID) and the information on the date and time (evaluation execution date and time).

The terminal apparatus 6 includes a communication unit 30, a display control unit 31, and an operation reception unit 32. Each of the functional units is implemented by the CPU 501 executing an instruction included in one or more programs installed in the terminal apparatus 6.

The communication unit 30 is a communication function of the terminal apparatus 6, and transmits and receives information to and from, for example, the analysis apparatus 5 via the communication network 4.

The display control unit 31 causes a screen of the terminal apparatus 6 to display a screen of the application for evaluating and managing a mental health status and the determination result of the mental health status of the user.

The operation reception unit 32 receives an operation such as inputting a character or pressing a button performed by the administrator who operates the terminal apparatus 6 via a keyboard or a pointing device of the terminal apparatus 6.

Overall Process Performed by Health Status Management System

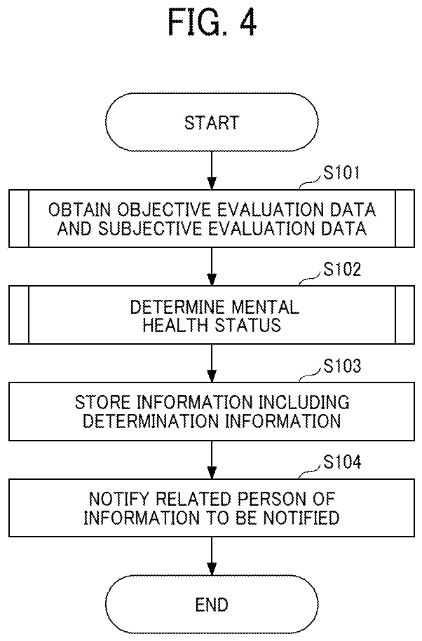

FIG. 4 is a flowchart of an overall process performed by the health status management system 1 according to the present embodiment. The processing steps in FIG. 4 are described below.

Step S101: The measurement unit 13 of the information processing apparatus 2 measures and obtains the pulse wave of the user as the objective evaluation data related to the mental health status of the user, by using the measurement device 3 such as a camera. In the health status management system 1, since the pulse wave can be measured in a non-contact manner from a moving image of the face of the user by using a camera. This allows the user to easily input a subjective value in the subjective evaluation by, for example, a mouse operation or a touch operation on a touch panel while measuring the pulse wave without being obstructed by the measurement device 3. Further, the acquisition unit 14 of the information processing apparatus 2 obtains the answer of the user to the question related to the mental health status as the subjective evaluation data. The question related to the mental health status may be, for example, “Do you feel you've been accumulating tiredness lately?” Details of the method for obtaining the objective evaluation data and the subjective evaluation data are described later. The communication unit 10 of the information processing apparatus 2 transmits the objective evaluation data and subjective evaluation data, which are obtained, to the communication unit 20 of the analysis apparatus 5. In the health status management system 1, a period of obtaining the objective evaluation data and the subjective evaluation data can be reduced, or shortened, by at least partially overlapping a period of obtaining the objective evaluation data and a period of obtaining the subjective evaluation data in Step S101.

Step S102: The communication unit 20 of the analysis apparatus 5 obtains the objective evaluation data and the subjective evaluation data that are related to the mental health status of the user from the communication unit 10 of the information processing apparatus 2. The determination unit 21 of the analysis apparatus 5 determines the mental health status of the user based on the objective evaluation data and the subjective evaluation data. For example, the determination unit 21 determines the mental health status of the user using binary evaluation of good and bad. Details of the process of determining the mental health status are described later. In the health status management system 1, the mental health status is determined by using both the objective evaluation data and the subjective evaluation data, so that the mental health status can be more accurately determined as compared with a case where the mental health status is determined by using one of the objective evaluation data and the subjective evaluation data.

Step S103: The storage unit 25 of the analysis apparatus 5 stores the objective evaluation data and the subjective evaluation data received by the communication unit 20 of the analysis apparatus 5, and the determination result of the mental health status determined by the determination unit 21. The storage unit 25 may store the objective indicator and the subjective indicator, which are described later, in alternative to the objective evaluation data and the subjective evaluation data. The management unit 22 of the analysis apparatus 5 causes the storage unit 25 to store, for example, the information identifying the user (for example, name and user ID) and the information on date and time (evaluation execution date and time) in association with the objective evaluation data, the subjective evaluation data, and the determination result. As described above, the management unit 22 integrates information related to mental health the status for each of all users to centrally manage the information.

Step S104: Based on the determination result determined by the determination unit 21 of the analysis apparatus 5 in Step S102, the notification unit 23 of the analysis apparatus 5 notifies the information to be notified to the administrator who manages, supervises, or observes the health status of the user. The information to be notified includes, for example, information identifying the user whose mental health status is determined to be bad in Step S102, and a recommendation for preventing the mental health status of the user from deteriorating. The recommendation for preventing the mental health status of the user from deteriorating is information useful for improving the mental health status of the user. The information useful for improving the mental health status is, for example, information on workstyles, exercise, food and supplement, sleep, massage/relaxation/mindfulness, healing/fatigue-relief items, environment, or counseling. The notification unit 23 notifies the information to be notified by transmitting to the communication unit 30 of the terminal apparatus 6 information for displaying the information to be notified on the screen of the terminal apparatus 6 operated by the administrator. For example, the notification unit 23 transmits, to the communication unit 30 of the terminal apparatus 6, information generated based on a data format requested by an OS native application or a web application running on the terminal apparatus 6 for displaying the information to be notified. Alternatively, the notification unit 23 may execute the notification by transmitting to an electronic mail (e-mail) address of the administrator an e-mail including the content of the information to be notified. Further, the notification unit 23 may transmit the notification to the communication unit 10 of the information processing apparatus 2 operated by the user in substantially the same procedure, or may execute the notification by transmitting to an e-mail address of the user an e-mail including the content of the information to be notified.

With the above-described process, the administrator can grasp a user to which attention should be paid, and thus can take measures at an early stage before the mental health status of the users deteriorates. Further, the administrator can take more effective measures to the user based on the notified recommendation for preventing the mental status of the user from deteriorating. In other words, by using the health status management system 1, symptoms of mental health disorder can be prevented. The details of the processing of Step S101 and the processing of Step S102 are described with reference to another flowchart.

Process for Obtaining Objective Evaluation Data and Subjective Evaluation Data

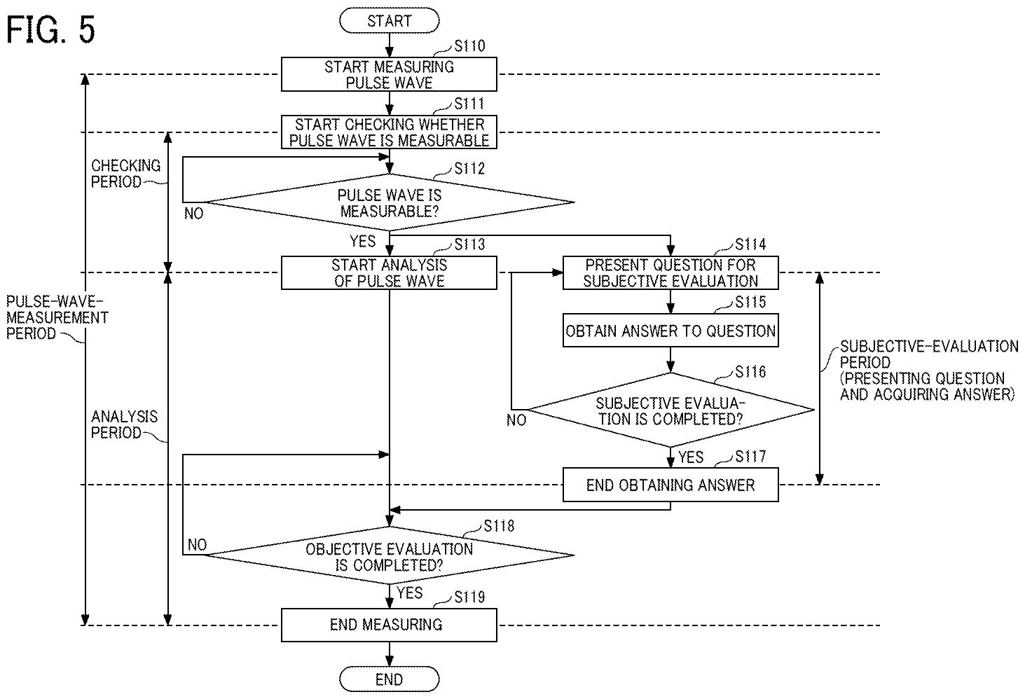

FIG. 5 is a flowchart of a process for obtaining the objective evaluation data and the subjective evaluation data according to the present embodiment. Details of the processing of Step S101 in FIG. 4 are described with reference to the flowchart of FIG. 5.

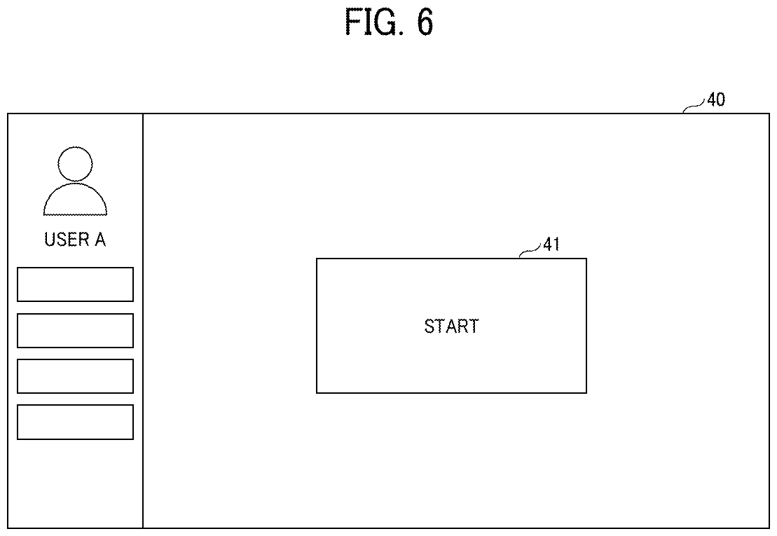

Step S110: The display control unit 11 of the information processing apparatus 2 causes a screen of the information processing apparatus 2 to display a screen of the application for evaluating a mental health status. FIG. 6 is a diagram illustrating a screen for starting the measurement according to the present embodiment. On a screen 40 of FIG. 6, a start button 41 to be pressed to start measuring a pulse wave is displayed. The operation reception unit 12 of the information processing apparatus 2 receives an operation of pressing the start button 41 performed by the user. The measurement unit 13 of the information processing apparatus 2 starts measuring the pulse wave in response to the operation of pressing of the start button 41. Alternatively, the measurement unit 13 of the information processing apparatus 2 may start measuring the pulse wave at a time when the application for evaluating a mental health status is activated. The control unit 15 of the information processing apparatus 2 sets the time at which the measurement unit 13 started measuring the pulse wave to the start time of a period of measuring the pulse wave. At this time, whether the pulse wave of the user is appropriately measured is not checked.

Returning to FIG. 5, the process is further described.

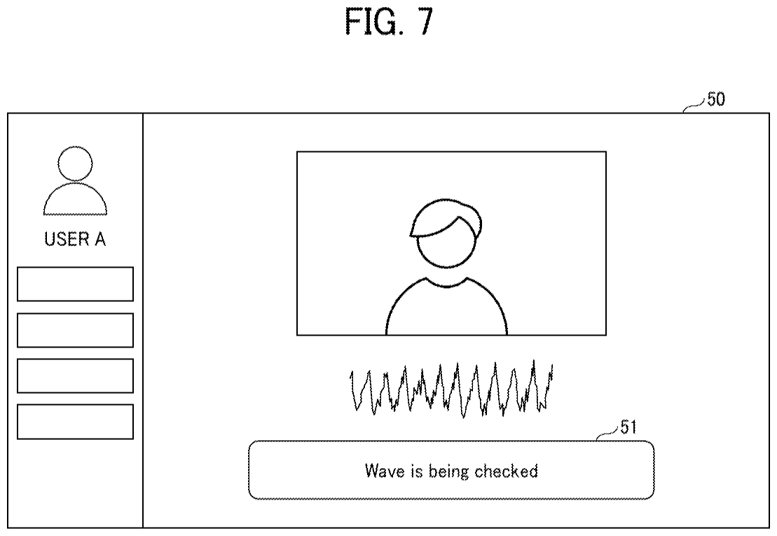

Step S111: The control unit 15 of the information processing apparatus 2 causes the measurement unit 13 of the information processing apparatus 2 to start checking whether the pulse wave is appropriately measurable, and sets the time at which the checking started to the start time of the period of checking whether the pulse wave is appropriately measurable. In the present embodiment, since the pulse wave is measured based on image information obtained by capturing the face of the user by a camera, whether the face of the user is appropriately captured with the camera is to be checked. The start of the checking is preferably after the face of the user is displayed on a display screen of the application. Further, since the signal value of the pulse wave greatly changes during calibration of the camera, the start of the checking is preferably after the completion of the calibration of the camera. FIG. 7 is a diagram illustrating a screen for checking pulse wave measurement according to the present embodiment. As illustrated in FIG. 7, the display control unit 11 of the information processing apparatus 2 causes a screen 50 to display a message 51, for example, “Wave is being checked,” after starting checking whether the pulse wave is measurable. Returning to FIG. 5, the process is further described.

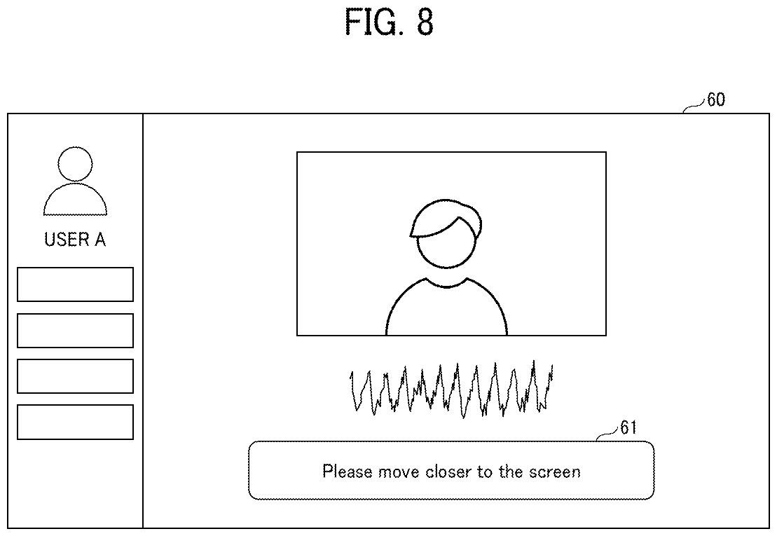

Step S112: The measurement unit 13 of the information processing apparatus 2 checks whether the pulse wave is appropriately measurable. The measurement unit 13 determines whether the pulse wave is appropriately measurable based on, for example, whether a position of the face of the user is within a predetermined imaging range of the camera, whether an RGB (Red Green Blue) value of the skin region in the captured image data is within a predetermined range, or whether a signal-to-noise ratio of the pulse wave is equal to or greater than a predetermined value. When the result of the checking indicates that the pulse wave is appropriately measurable, the control unit 15 of the information processing apparatus 2 transitions the process to Step S13 and Step S114. When the result of the checking indicates that the pulse wave is not appropriately measurable, namely a failure to measure the pulse wave appropriately, the display control unit 11 of the information processing apparatus 2 displays on the screen a warning (alert) for notifying the user that the pulse wave is not appropriately measurable. In addition to information indicating that the pulse wave is not appropriately measurable, namely a failure to measure the pulse wave appropriately, the warning (alert) preferably provides information on a measure to be taken to measure the pulse wave appropriately. FIG. 8 is a diagram illustrating a screen for displaying a warning according to the present embodiment. As illustrated in FIG. 8, the display control unit 11 of the information processing apparatus 2 causes a screen 60 to display a message 61, for example, “Please move closer to the screen,” as the information on a measure to be taken to measure the pulse wave appropriately. As described above, in case where the pulse wave is not appropriately measurable due to, for example, body movement of the user, a warning (alert) is displayed to correct the state of the user, and failure in measuring the pulse wave can be prevented, accordingly. As a result, even when the objective evaluation and the subjective evaluation are performed simultaneously, it is possible to prevent the total evaluation period from becoming long and to accurately evaluate the mental health status of the user. Returning to FIG. 5, the process is further described.

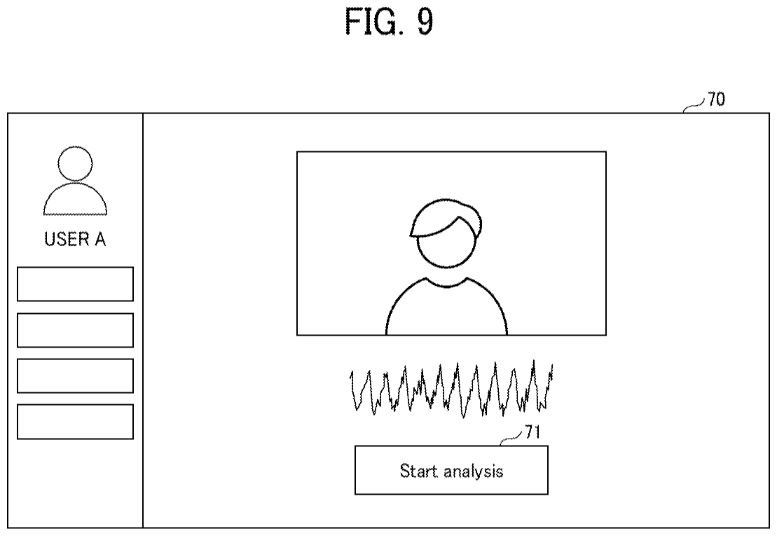

Step S113: The control unit 15 of the information processing apparatus 2 causes the measurement unit 13 of the information processing apparatus 2 to start analyzing the pulse wave, and sets the time at which the analysis is started to the start time of the period of analyzing. The measurement unit 13 of the information processing apparatus 2 starts analyzing the pulse wave. For example, the display control unit 11 of the information processing apparatus 2 displays an evaluation-start button on a screen of the application for evaluating a mental health status. The operation reception unit 12 of the information processing apparatus 2 receives an operation of pressing the evaluation-start button performed by the user. The control unit 15 sets a time at which the user clicks the evaluation-start button displayed on the screen of the application for evaluating a mental health status to the start time of the period of analyzing the pulse wave, and the measurement unit 13 starts analyzing the pulse wave. FIG. 9 is a diagram illustrating a screen for starting analyzing a pulse wave according to the present embodiment. On a screen 70 of FIG. 9, an analysis-start button 71 to be pressed to start analyzing a pulse wave is displayed. Alternatively, the control unit 15 may cause the measurement unit 13 to start analyzing the pulse wave at a time when the measurement unit 13 checks, or confirms, that the pulse wave is appropriately measurable in Step S113, and may set the time at which the analysis is started to the start time of the period of analyzing. As described above, in the health status management system 1, since analyzing the pulse wave is started after checking, or confirming, that the pulse wave is appropriately measurable in Step S112, even when the objective evaluation and the subjective evaluation are performed simultaneously, the risk of failure in measuring the pulse wave after the starting analyzing the pulse wave can be reduced. Returning to FIG. 5, the process is further described.

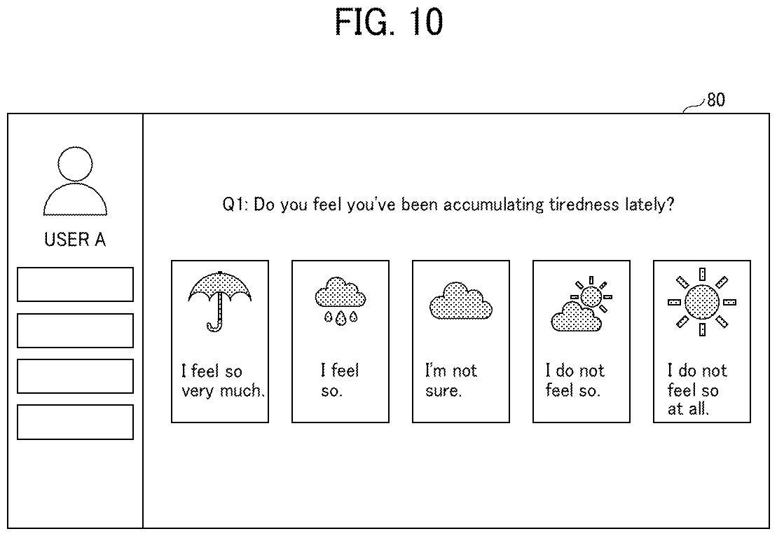

Step S114: The control unit 15 of the information processing apparatus 2 causes the display control unit 11 of the information processing apparatus 2 to display a question for the subjective evaluation on the screen (presents the question to the user), and sets the time at which the question is started being displayed to the start time of the period of performing the subjective evaluation (presenting the one or more questions and obtaining the one or more answers). The display control unit 11 of the information processing apparatus 2 displays the question for the subjective evaluation on the screen of the application for evaluating a mental health status. FIG. 10 is a diagram illustrating a screen for displaying a question for subjective evaluation according to the present embodiment. On a screen 80 of FIG. 10, a question related to the mental health status and answer buttons corresponding to answer candidates to the question are displayed. The question is, for example, “Do you feel you've been accumulating tiredness lately?” and the answer buttons indicate, for example, “I feel so very much,” “I feel so,” “I'm not sure,” “I do not feel so,” and “I do not feel so at all.” The question may be any question as long as asking about the mental health, for example, a question asking about stress, lack of sleep, work satisfaction, or personal relationship. Further, the method of obtaining the answer is not limited to the method in which the user selects an answer candidate for the question, and may be, for example, a visual analogue scale (VAS) method in which the user answers about the current health status by positioning, for example, a mark on a scale, or a method in which the user freely enters text as the answer. As illustrated in FIG. 10, the display control unit 11 displays the one or more questions one by one on the screen of the application for evaluating a mental health status. In case where multiple questions are displayed on a single screen, the size of each of the characters of the questions is small. Due to this reason, the number of questions displayed on a single screen is preferably less, and preferably one on a single screen. Displaying one question on one screen can reduce the body motion of the user of moving closer to the screen to see small characters. This can reduce the noise caused by the body motion and prevent the measurement accuracy of the pulse wave from being low.

Further, in the same region of the screen of the application for evaluating a mental health status, the display control unit 11 displays the screen related to the objective evaluation in Steps S110 to S113 and then the switch the screen to display the screen related to the subjective evaluation in Step S114. In other words, the display control unit 11 displays, on the screen, the information on the objective evaluation alone before the start time of the period of performing subjective evaluation, and displays, on the screen, the information on the subjective evaluation alone after the start time of the period of performing the subjective evaluation. As described above, the screens of the objective evaluation and the subjective evaluation are not displayed at the same time even in different regions. By so doing, the body motion due to the movement of the line of sight and the posture of the user can be reduced.

By preventing failure in measuring the pulse wave, even when the objective evaluation and the subjective evaluation are performed simultaneously, it is possible to prevent the total evaluation period from becoming long and to accurately evaluate the mental health status of the user. Returning to FIG. 5, the process is further described.

Step S115: The operation reception unit 12 of the information processing apparatus 2 receives, for example, an operation performed by the user of pressing an answer button in response to the question related to the mental health status illustrated in FIG. 10. The acquisition unit 14 of the information processing apparatus 2 obtains the content received by the operation reception unit 12 as a response to the subjective evaluation by the user.

Step S116: The control unit 15 of the information processing apparatus 2 determines whether to end the subjective evaluation. For example, the control unit 15 determines to end the subjective evaluation when the acquisition unit 14 obtains answers corresponding to the number of questions (minimum number of questions) determined in advance, and otherwise determines not to end the subjective evaluation. Further, the control unit 15 transitions the process to Step S117 after determining to end the subjective evaluation, or transitions the process to Step S114 after determining not to end the subjective evaluation. The minimum number of questions is set in advance as the minimum number of questions used for performing the subjective evaluation. The minimum number is set by, for example, the administrator who manages, supervises, or observes the health status of the user.

The minimum number of questions may be, for example, five questions or one question. As the number of questions increases, the mental health status can be determined more accurately, but the time taken for the answering the questions increases. Due to this, the minimum number of questions is preferably set such that corresponding answers can be obtained within the minimum measurement period for the pulse wave described later. As a result, the total evaluation period in relation to the mental health status can be reduced, or shortened, and the burden on the user can be reduced, accordingly.

Alternatively, when the control unit 15 may perform control so as to obtains answers to the questions of which the number is more than the minimum number of questions (in other words, increases the number of questions) in case where the minimum measurement period for the pulse wave described later has not been elapses before a time when the answers to the question of which the number is the minimum number of questions are obtained, or in case where a certain period of time remains before the end time of the minimum measurement period. Accordingly, the mental health status without can be determined more accurately without extending the period taken for the entire evaluation in relation to the mental health status.

Step S117: The control unit 15 of the information processing apparatus 2 causes the acquisition unit 14 of the information processing apparatus 2 to end the subjective evaluation (obtaining the one or more answers) and sets the current time as the end time of the subjective evaluation (presenting the one or more question and obtaining the corresponding one or more answers).

Step S118: The control unit 15 of the information processing apparatus 2 determines whether to end the objective evaluation. When determining to end the objective evaluation, the control unit 15 transitions the process to Step S119. When determining not to end the objective evaluation, the control unit 15 repeats the processing of Step S118. For example, the control unit 15 determines whether to end the objective evaluation based on whether the minimum measurement period for the pulse wave determined in advance (may be simply referred to as a minimum measurement period) has elapsed and whether the subjective evaluation has ended. For example, when the minimum measurement period has elapsed and the subjective evaluation has ended, the control unit 15 determines to end the objective evaluation. When the subjective evaluation has ended, but the minimum measurement period has not elapsed, the control unit 15 does not determine to end the objective evaluation until the minimum measurement period elapses.

When the subjective evaluation has not ended at the time when the minimum measurement period has elapsed, the control unit 15 extends the period of measuring the pulse wave and does not end the objective evaluation until the subjective evaluation ends. As described above, when the subjective evaluation is not ended, the period for measuring is extended to extend the objective evaluation to be executed. By so doing, the accuracy of the objective evaluation can increase. The reason for this is that the longer the period of measuring the pulse wave is, the longer the period of obtaining the time average is, so that the influence of noise is reduced and the measurement accuracy increases.

The minimum measurement period is a minimum period for measuring the pulse wave used to determine the mental health status. Although the minimum measurement period varies depending on the determination method of the mental health status, the minimum measurement period for the pulse wave to be used to determine the mental health status is typically, for example, about 5 seconds to 5 minutes. Further, there is a trade-off relationship between the measurement period and the reliability of the measurement result. The shorter the measurement period is, the lower the reliability of the measurement result becomes, and the longer the measurement period is, the higher the reliability of the measurement result becomes. Accordingly, an optimum measurement period is preferably set according to the purpose of evaluation.

Step S119: The control unit 15 of the information processing apparatus 2 causes the measurement unit 13 of the information processing apparatus 2 to end analyzing the pulse wave, and sets the current time to the end time of the period of performing the objective evaluation, the end time of the period of measuring the pulse wave, and the end time of the period of analyzing.

According to the above-described process, in the health status management system 1, the measurement unit 13 of the information processing apparatus 2 obtains the objective evaluation data, and the acquisition unit 14 of the information processing apparatus 2 obtains the subjective evaluation data, in relation to the mental health status of the user. In the health status management system 1 according to the present embodiment, the total evaluation period can be reduced, or shortened, by overlapping the period for performing the objective evaluation and the period for performing the subjective evaluation.

The control unit 15 of the information processing apparatus 2 controls the start time (S110) and the end time (S119) of the period of measuring the pulse wave, the start time (S111) and the end time (S112) of the period of checking whether the pulse wave is appropriately measurable, and the start time (S113) and the end time (S119) of the period of analyzing the pulse wave, through time control. Further, the control unit 15 controls the start time of the period for performing the subjective evaluation (S114, display time of the first question) and controls the end time of the period of performing the subjective evaluation (S117, acquisition time of the last answer) through time control.

Further, the control unit 15 of the information processing apparatus 2 controls the start time of the period of measuring the pulse wave to be earlier than the start time of the period of performing the subjective evaluation (the display time of the first question) through time control. Alternatively, the control unit 15 controls the start time of the period of analyzing the pulse wave to be earlier than or equal to (same as) the start time of the period of performing the subjective evaluation (the display time of the first question) through time control. Accordingly, since the subjective evaluation can be started after checking that measuring the pulse wave is appropriately performed, the risk of failure in measuring the pulse wave can be reduced. Accordingly, the total evaluation period can be reduced, or shortened, and the burden on the user can be reduced.

The control unit 15 of the information processing apparatus 2 sets the period of measuring the pulse wave in the objective evaluation to be a predetermined minimum measurement period, and sets the number of questions in the subjective evaluation to be a predetermined minimum number of questions. For example, by setting the minimum measurement period to a typically-known minimum value (40 seconds to 90 seconds) and the minimum number of questions to one in advance, the total evaluation period can be reduced, or shortened, to the minimum value, and the burden on the user can be reduced. Further, the control unit 15 controls the end time of the period of measuring the pulse wave to be later than or equal to (same as) the end time of the period of performing the subjective evaluation (the acquisition time of the last answer) through time control. By so doing, measuring the pulse wave does not end before the subjective evaluation ends, and the period of measuring the pulse wave is extended as much as possible. Accordingly, the measurement accuracy of the pulse wave can be increased.

Process for Measuring Pulse Wave in Objective Evaluation

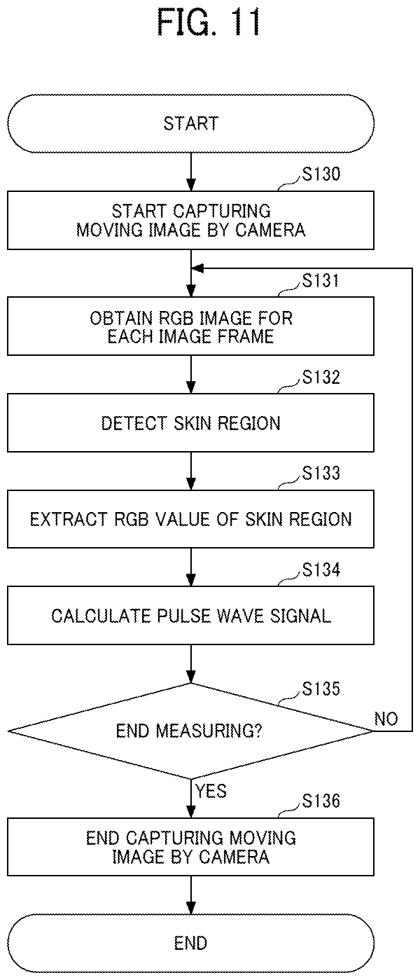

FIG. 11 is a flowchart of a process for measuring a pulse wave according to the present embodiment. Details of the process for measuring the pulse wave in the objective evaluation in case where an RGB (Red, Green, Blue) camera is used as the measurement device 3 of the information processing apparatus 2 are described with reference to FIG. 11. The processing steps in FIG. 11 are described below.

Step S130: The measurement unit 13 of the information processing apparatus 2 starts capturing a moving image by the RGB camera. The RGB camera obtains information regarding three colors of R (Red), G (Green), and B (Blue) (for example, 8 bits) per pixel. The settings for moving-image capturing includes a resolution of 640×480 pixels and a frame rate of 30 frames per second (FPS). This settings may be any setting as long as the pulse wave can be measured, but typically, the resolution of 640×480 pixels or more and the frame rate of 30 FPS or more are preferably used.

Step S131: The measurement unit 13 of the information processing apparatus 2 obtains an RGB image for each image frame based on the setting set in Step S130.

Step S132: The measurement unit 13 of the information processing apparatus 2 detects the skin region of the user based on the RGB image obtained in Step S131. For example, the measurement unit 13 detects coordinate positions of feature points such as the eyes, the nose, and the mouth of the user by a face detection technique, and detects the skin region of face based on the detected coordinate positions. The skin region includes preferably at least a cheek portion. The cheek portion is a region of the face where a pulse wave signal is easily detected. Accordingly, the pulse wave signal can be calculated more accurately by detecting the skin region including a cheek portion. As the method of detecting a skin region, another method may be used as long as the skin region can be detected. For example, pixels whose RGB values are within a range of RGB values of a general skin color may be extracted, and a set of the pixels may be detected as the skin region. Alternatively, the user may input coordinate information of the skin region while viewing a camera image displayed on a screen of the application, and the measurement unit 13 may detect the skin region based on the input coordinate information. The measurement unit 13 may detect the skin region by dividing the skin region into multiple regions. For example, the measurement unit 13 may detect the skin region by dividing the skin region into multiple regions such as a region including the left cheek, another region including the right cheek, and still another region including the nose.

Step S133: The measurement unit 13 of the information processing apparatus 2 extracts the RGB values of the pixels included in the skin region detected in Step S132. For example, the measurement unit 13 extracts, as an RGB value of the skin region, a value obtained by averaging the R values, the G values, and the B values of the pixels included in the skin region. By using the averaged RGB value, the influence of noise on the pulse wave can be reduced, and the pulse wave signal can be calculated more accurately. As the method of extracting an RGB value, another method may be used as long as the RGB value including information of the pulse wave can be extracted. For example, the measurement unit 13 may extract a median value of each of the R value, the G value, and the B value of the pixel included in the skin region as an RGB value of the skin region.

Step S134: The measurement unit 13 of the information processing apparatus 2 calculates a pulse wave signal based on the RGB value of the skin region extracted in Step S132. For example, the measurement unit 13 calculates the pulse wave signal that is a signal change component associated with a pulse, based on time-series data of RGB value of the skin region using a method of independent principal component analysis. The pulse wave signal is time-series data in which a signal value periodically changes according to the pulse. As the method of calculating a pulse wave signal, another method may be used as long as the pulse wave component can be obtained. For example, the measurement unit 13 may use, as the pulse wave signal, the G value that contains the largest amount of pulse wave components, or may use, as the pulse wave signal, a value obtained by taking a difference or a ratio between the G value and one of the R value or the B value.

Step S135: The control unit 15 of the information processing apparatus 2 determines whether to end measuring the pulse wave. When the control unit 15 determines to end measuring the pulse wave, the control unit 15 transitions the process to Step S136. When the control unit 15 determines not to end measuring the pulse wave, the control unit 15 transitions the process to Step S131.

Step S136: The measurement unit 13 of the information processing apparatus 2 ends capturing the moving image by the RGB camera.

Process for Determining Mental Health Status

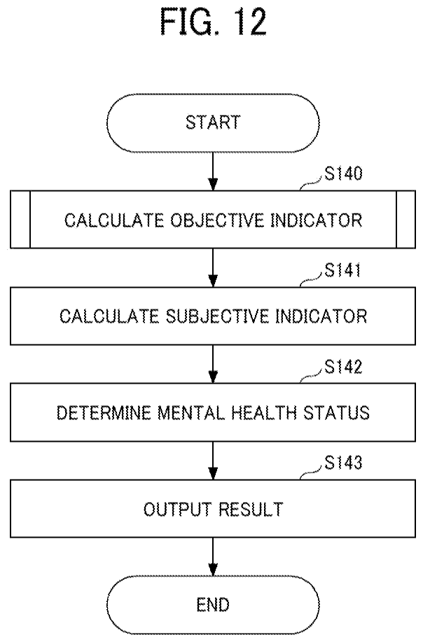

FIG. 12 is a flowchart of a process for determining a mental health status according to the present embodiment. In the flowchart, the analysis apparatus 5 determines the mental health status of the user based on the objective evaluation data and the subjective evaluation data received from the information processing apparatus 2. The processing steps in FIG. 12 are described below.

Step S140: The communication unit 10 of the information processing apparatus 2 transmits, to the communication unit 20 of the analysis apparatus 5, the objective evaluation data obtained by measuring the pulse wave described in the flowcharts of FIGS. 5 and 11. The calculation unit 24 of the analysis apparatus 5 calculates an objective indicator based on the objective evaluation data received by the communication unit 20 of the analysis apparatus 5. For example, the calculation unit 24 calculates Total Power (TP) that is an autonomic nervous system indicator based on the pulse wave signal in the objective evaluation data, and uses the TP as the objective indicator. Details of the process for calculating the TP, which is an autonomic nervous system indicator, based on a pulse wave signal are described later. The TP is an indicator associated with the accumulation of fatigue, and it is known that the accumulation of fatigue leads to disorders of mental health status such as loss of motivation or depressive tendency. Accordingly, by using the TP as an objective indicator, an objective mental health status can be evaluated. Another indicator may be used as the objective indicator calculated by the calculation unit 24 as long as the indicator indicates a mental health status. For example, the calculation unit 24 may use average heart rate (HR), Standard Deviation of Normal-to-Normal intervals (SDNN), Low Frequency (LF), which is an indicator mainly indicating activity of sympathetic nervous system, High Frequency (HF), which is an indicator indicating activity of parasympathetic nervous system, LF/HF, which is an indicator indicating balance of autonomic nerve system function, or Ln(TP), which is a natural logarithm of TP. The number of objective indicators to be calculated is not limited to one, and multiple indicators may be used. For example, the calculation unit 24 may use both TP and LF/HF that are autonomic nervous system indicators as the objective indicators. The calculation unit 24 may calculate the objective indicator based on past objective evaluation data in addition to the current objective evaluation data. For example, the calculation unit 24 may use, as an objective indicator, an average value of the TP in the most recent one month or an amount of change from the previously calculated TP.

Step S141: The communication unit 10 of the information processing apparatus 2 transmits, to the communication unit 20 of the analysis apparatus 5, the subjective evaluation data obtained in the subjective evaluation described in the flowchart of FIG. 5. The calculation unit 24 of the analysis apparatus 5 calculates a subjective indicator based on the subjective evaluation data received by the communication unit 20 of the analysis apparatus 5. For example, based on the subjective evaluation data, the calculation unit 24 calculates a numerical value of fatigue (1: very tired, 2: tired, 3: neither, 4: not tired, 5: not tired at all) as a subjective indicator. Since it is known that a level of feeling of fatigue, which is a subjective value of fatigue, is also an indicator for determining a mental health status, a subjective mental health status can be evaluated by using the numerical value of fatigue as a subjective indicator. Another indicator may be used as the subjective indicator calculated by the calculation unit 24 as long as the indicator indicates a mental health status. For example, the calculation unit 24 may use an indicator obtained by quantifying answers to the questions asking, for example, a feeling of stress or lack of sleep. The subjective indicator to be calculated does not need to be one, and multiple indicators may be used. For example, the calculation unit 24 may use both the numerical value for the feeling of fatigue and a numerical value for the feeling of stress as subjective indicators. The calculation unit 24 may calculate the subjective indicator based on past subjective evaluation data in addition to the current subjective evaluation data. For example, the calculation unit 24 may use, as a subjective indicator, an average value of the numerical values of the feeling of fatigue in the most recent one month or an amount of change from the numerical value of the feeling of fatigue in the previous time. Further, the calculation unit 24 may perform the calculation of the objective indicator in Step S140 and the calculation of the subjective indicator in Step S141 in reverse order.

Step S142: The determination unit 21 of the analysis apparatus 5 determines the mental health status of the user based on the objective indicator calculated by the calculation unit 24 of the analysis apparatus 5 in Step S140 and the subjective indicator calculated in Step S141. For example, when “the numerical value of the feeling of fatigue as a subjective indicator is less than or equal to a first reference value” or “the numerical value of the feeling of fatigue as a subjective indicator is greater than the first reference value but the TP as an objective indicator is less than or equal to a second reference value,” the determination unit 21 determines that the mental health status is bad and gives an instruction to issue a warning (alert). In the present embodiment, an appropriate value is set to each of the first reference value and the second reference value. A user having a high subjective fatigue level typically has a high objective fatigue level. Accordingly, with the condition of “the numerical value of the feeling of fatigue as a subjective indicator is less than or equal to a first reference value,” users in bad mental health statuses can be determined. Further, a user having a low subjective fatigue level may have a high objective fatigue level. Accordingly, with the condition of “the numerical value of the feeling of fatigue as a subjective indicator is greater than the first reference value but the TP as an objective indicator is less than or equal to a second reference value,” users in bad mental health statuses can be determined. Further, the mental health status may be determined by another method as long as the mental health status of the user can be determined. For example, the mental health status may be determined by creating an evaluation formula using the objective indicator and the subjective indicator as parameters and comparing the calculated evaluation value with a predetermined reference value. Alternatively, the mental health status may not be determined using the binary evaluation of good/bad, but using multilevel evaluation such as, for example, ternary evaluation of good/caution/warning. Further, the reference value may be changed by the administrator. Accordingly, it is possible to control the number of users to be notified of a warning (alert) in accordance with the situation, for example, by setting the reference value to be strict so that all users in a bad mental health status can be extracted, or conversely setting the reference value to be gentle so that the users in a particularly bad mental health status are extracted.

Step S143: The notification unit 23 of the analysis apparatus 5 notifies a result indicating the mental health status determined in Step S142 by transmitting the result to the communication unit 30 of the terminal apparatus 6 used by the administrator. The display control unit 31 of the terminal apparatus 6 displays, on a screen, the received result indicating the mental health status. Further, the notification unit 23 of the analysis apparatus 5 may transmit the result indicating the mental health status determined in Step S142 to the communication unit 10 of the information processing apparatus 2 used by the user to notify the result to the user. The display control unit 11 of the information processing apparatus 2 displays, on a screen, the received result of the mental health status. Accordingly, the administrator and the user can check the result of the notified mental health status. Alternatively, the notification unit 23 may perform the notification by transmitting an electronic mail including the result of the mental health status to the mail addresses of the administrator and the user.

With the above-described process, the health status management system 1 can determine the mental health status of the user based on the objective evaluation data and the subjective evaluation data.

Process for Calculating Objective Indicator

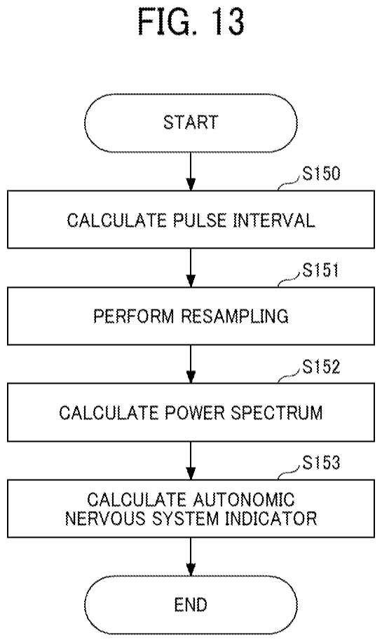

FIG. 13 is a flowchart of a process for calculating an objective indicator according to the present embodiment. Details of the process for calculating the objective indicator corresponding to the processing of Step S140 in FIG. 12 are described with reference to the flowchart of FIG. 13. The processing steps in FIG. 13 are described below.

Step S150: The calculation unit 24 of the analysis apparatus 5 calculates a pulse interval based on the pulse wave signal. The pulse interval may be calculated by using a known technique. For example, the calculation unit 24 detects a time at which the pulse wave signal reaches a peak, and calculates a pulse interval based on a time interval between adjacent peaks.

Step S151: The calculation unit 24 of the analysis apparatus 5 performs resampling in a manner that the time intervals of the time-series data for the pulse intervals calculated in Step S150 become evenly spaced. The time interval of the resampling is, for example, 0.25 seconds. The signal value at the elapsed time to be resampled may be obtained by, for example, interpolation. As an interpolation method, for example, linear interpolation or spline interpolation can be used.

Step S152: The calculation unit 24 of the analysis apparatus 5 calculates a power spectrum based on the time-series data for the pulse intervals calculated in Step S151. As a method of calculating the power spectrum, a known frequency analysis method can be used. The calculation unit 24 calculates power at a designated frequency by, for example, a maximum entropy method. Parameters such as lag in the calculation of the power are set to appropriate values.

Step S153: The calculation unit 24 of the analysis apparatus 5 calculates an autonomic nervous system indicator based on the power spectrum calculated in Step S152. As the autonomic nervous system indicator, for example, an LF/HF value representing the balance of the autonomic nervous system function or a TP value representing the work of the entire autonomic nervous system function can be used. The LF/HF value and the TP value may be calculated using a known method. For example, the calculation unit 24 can calculate the LF/HF value by taking the ratio of the power integration value in the frequency band of 0.04 to 0.15 [Hz] as the LF value and the power integration value in the frequency band of 0.15 to 0.40 [Hz] as the HF value, and calculate the TP value by taking the sum of the two values.

Through the above processing, the calculation unit 24 of the analysis apparatus 5 calculates the objective indicator.

In the evaluation of the mental health status of the health status management system 1, the total evaluation period can be reduced, or shortened, by overlapping the period of performing the objective evaluation with the period of performing the subjective evaluation. Further, reducing the total evaluation period can reduce the burden on the user.

Embodiments of the present disclosure are not limited to the above-described examples, and various modifications and replacements are possible within the scope not departing from the gist of the present disclosure.

For example, the configuration example of the functional block diagram of FIG. 3 is divided according to the main functions in order to facilitate understanding of the processing by the information processing apparatus 2, the analysis apparatus 5, and the terminal apparatus 6 that are included in the health status management system 1. No limitation to a scope of the present disclosure is intended by how the processes are divided or by the name of the processes. The processing implemented by the information processing apparatus 2, the analysis apparatus 5, or the terminal apparatus 6 may be divided into a greater number of units depending on the processing details. Further, a single processing unit can be further divided into multiple processing units.

Each of the functions of the described embodiments may be implemented by one or more processing circuits or circuitry. In the description of the above-described embodiments, the “processing circuit or circuitry” in the present specification includes a programmed processor to execute each function by software, such as a processor implemented by an electronic circuit, and devices, such as an application specific integrated circuit (ASIC), a digital signal processors (DSP), a field programmable gate array (FPGA), and conventional circuit modules arranged to perform the recited functions.

The apparatuses and devices described above are serves as one of several computing environments for implementing the above-described embodiment. In some embodiments, the information processing apparatus 2, the analysis apparatus 5, or the terminal apparatus 6 may include multiple computing devices such as server clusters. The computing devices are configured to communicate with each other via any type of communication link, including a network, shared memory, etc., and perform the processes disclosed in the above-described embodiment.

Aspects of the present disclosure are, for example, as follows.

Aspect 1

A health status management system includes an information processing apparatus operated by a user and an analysis apparatus that can communicate with the information processing apparatus. The information processing apparatus includes a measurement unit to measure a pulse wave of the user to obtain objective evaluation data for objective evaluation in relation to a mental health status, an acquisition unit to obtain one or more answers of the user to one or more questions related to the mental health status as subjective evaluation data for subjective evaluation in relation to the mental health status, a control unit to control a period of measuring the pulse wave and a period of performing the subjective evaluation to overlap with each other and to control a start time of the period of measuring the pulse wave to be earlier than a start time of the period of performing the subjective evaluation, a communication unit to transmit the objective evaluation data and the subjective evaluation data to an analysis apparatus, and a display control unit to display, on a screen, information on the objective evaluation and information on the subjective evaluation.

Aspect 2

In the health status management system of Aspect 1, the measurement unit starts analyzing the measured pulse wave after checking that the pulse wave is appropriately measurable, and the control unit controls a start time of a period of analyzing the pulse wave to be earlier than or same as the start time of the period of performing the subjective evaluation.

Aspect 3

In the health status management system of any one of Aspect 1 and Aspect 2, the control unit sets the period of measuring the pulse wave to a minimum measurement period, and a number of questions corresponding to the one or more questions in the subjective evaluation to be a minimum number, and controls an end time of the period of measuring the pulse wave to be later or same as an end time of the period of performing the subjective evaluation.

Aspect 4

In the health status management system of Aspect 3, the control unit extends the period of measuring the pulse wave in a case that obtaining the one or more answers to the one or more questions corresponding to the minimum number of questions by the acquisition unit is incomplete at a time when the minimum measurement period has elapsed.

Aspect 5

In the health status management system of any one of Aspect 3 and Aspect 4, the control unit increases the number of questions in a case that the one or more answers to the one or more questions corresponding to the minimum number of questions are obtained by the acquisition unit before the minimum measurement period has elapsed.

Aspect 6

In the health status management system of any one of Aspect 1 to Aspect 5, the display control unit displays the information on the objective evaluation alone on the screen before the start time of the period of performing the subjective evaluation, and displays the information on the subjective evaluation alone on the screen after the start time of the period of performing the subjective evaluation.

Aspect 7

In the health status management system of Aspect 6, the display control unit displays, on the screen, a warning in response to the measurement unit confirming a failure to measure the pulse wave appropriately.

Aspect 8

In the health status management system of any one of Aspect 3 and Aspect 4, the display control unit displays, on the screen, the one or more questions one by one in the subjective evaluation.

Aspect 9

In the health status management system of any one of Aspect 1 to Aspect 8, the measurement unit measures the pulse wave using a camera.

Aspect 10

In the health status management system of any one of Aspect 1 to Aspect 9, the analysis apparatus includes a determination unit to determine the mental health status of the user to obtain a determination result based on the objective evaluation data and the subjective evaluation data, and a storage unit to store, in a memory, the objective evaluation data, the subjective evaluation data, and the determination result.

Aspect 11

In the health status management system of Aspect 10, the communication unit notifies the administrator of information identifying the user and information for preventing the mental health status of the user from deteriorating, based on the determination result.

According to one or more embodiments, a non-transitory recording medium storing a plurality of instructions which, when executed by one or more processors, causes the processors to perform a method. The method includes measuring a pulse wave of a user to obtain objective evaluation data for objective evaluation in relation to a mental health status, obtaining an answer of the user to a question related to the mental health status as subjective evaluation data for subjective evaluation in relation to the mental health status, controlling a period of measuring the pulse wave and a period of performing the subjective evaluation to overlap with each other, and controlling a start time of the period of measuring the pulse wave to be earlier than or same as a start time of the period of performing the subjective evaluation.

In related art, since subjective evaluation is performed after objective evaluation in evaluating a mental health status, the total evaluation period is lengthy.

The longer the evaluation period is, the more the burden on a user becomes.

According to one or more embodiments of the present disclosure, the total period for evaluation including objective evaluation and subjective evaluation related to a mental health status can be reduced, or shortened.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application specific integrated circuits (ASICs), digital signal processors (DSPs), field programmable gate arrays (FPGAs), conventional circuitry and/or combinations thereof which are configured or programmed to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

1. A health status management system, comprising
circuitry configured to:
measure a pulse wave of a user to obtain objective evaluation data for objective evaluation in relation to a mental health status;
obtain one or more answers of the user to one or more questions related to the mental health status as subjective evaluation data for subjective evaluation in relation to the mental health status;
control a period of measuring the pulse wave and a period of performing the subjective evaluation to overlap with each other;
control a start time of the period of measuring the pulse wave to be earlier than a start time of the period of performing the subjective evaluation;
transmit, to additional circuitry, the objective evaluation data and the subjective evaluation data; and
display, on a screen, information on the objective evaluation and information on the subjective evaluation.

2. The health status management system of claim 1, wherein the circuitry is further configured to:
start analyzing the measured pulse wave after checking that the pulse wave is appropriately measurable; and
control a start time of a period of analyzing the pulse wave to be earlier than or same as the start time of the period of performing the subjective evaluation.

3. The health status management system of claim 2, wherein the circuitry is further configured to:
control the period of measuring the pulse wave to be a minimum measurement period, and a number of questions corresponding to the one or more questions in the subjective evaluation to be a minimum number; and
control an end time of the period of measuring the pulse wave to be later or same as an end time of the period of performing the subjective evaluation.

4. The health status management system of claim 3, wherein the circuitry is further configured to
extend the period of measuring the pulse wave, in a case that obtaining the one or more answers to the one or more questions corresponding to the minimum number of questions is incomplete at a time when the minimum measurement period has elapsed.

5. The health status management system of claim 3, wherein the circuitry is further configured to
increase the number of questions, in a case that the one or more answers to the one or more questions corresponding to the minimum number of questions are obtained before the minimum measurement period elapses.

6. The health status management system of claim 1, wherein the circuitry is configured to:
display the information on the objective evaluation alone on the screen before the start time of the period of performing the subjective evaluation; and
display the information on the subjective evaluation alone on the screen after the start time of the period of performing the subjective evaluation.

7. The health status management system of claim 6, wherein the circuitry is further configured to display, on the screen, a warning in response to confirming a failure to measure the pulse wave appropriately.

8. The health status management system of claim 1, wherein the circuitry is further configured to
display, on the screen, the one or more questions one by one in the subjective evaluation.

9. The health status management system of claim 1, wherein the circuitry is configured to
measure the pulse wave using a camera.

10. The health status management system of claim 1, further comprising the additional circuitry configured to operate in cooperation with the circuitry,
the additional circuitry being configured to:
determine, based on the objective evaluation data and the subjective evaluation data, the mental health status of the user being a first user to obtain a determination result;
store, in a memory, the objective evaluation data, the subjective evaluation data, and the determination result; and
transmit, to a terminal apparatus used by a second user, the determination result to notify the second user of the determination result, the second user being an administrator who manages the first user.

11. The health status management system of claim 10, wherein the additional circuitry is configured to:
transmit, to the terminal apparatus to notify the second user, information identifying the first user and information indicating a way of preventing the mental health status of the first user from deteriorating, based on the determination result.

12. An information processing apparatus, comprising circuitry configured to:
measure a pulse wave of a user to obtain objective evaluation data for objective evaluation in relation to a mental health status of the user;
obtain an answer of the user to a question related to the mental health status as subjective evaluation data for subjective evaluation in relation to the mental health status;
control a period of measuring the pulse wave and a period of performing the subjective evaluation to overlap with each other; and
control a start time of the period of measuring the pulse wave to be earlier than or same as a start time of the period of performing the subjective evaluation.

13. An information processing method, comprising:
measuring a pulse wave of a user to obtain objective evaluation data for objective evaluation in relation to a mental health status;
obtaining an answer of the user to a question related to the mental health status as subjective evaluation data for subjective evaluation in relation to the mental health status;
controlling a period of measuring the pulse wave and a period of performing the subjective evaluation to overlap with each other; and
controlling a start time of the period of measuring the pulse wave to be earlier than or same as a start time of the period of performing the subjective evaluation.
